SEQUENCE LISTING (10 sequences)

SEQ ID NO: 1 - DNA, 35 bp, Artificial Sequence
  ACA1-A5
acactctttccctacacgacgctcttccgatctat

SEQ ID NO: 2 - DNA, 35 bp, Artificial Sequence
  ACA1-A7
tagatcggaagagcacacgtctgaactccagtcac

SEQ ID NO: 3 - DNA, 35 bp, Artificial Sequence
  ACA2-A5
acactctttccctacacgacgctcttccgatctgt

SEQ ID NO: 4 - DNA, 35 bp, Artificial Sequence
  ACA2-A7
cagatcggaagagcacacgtctgaactccagtcac

SEQ ID NO: 5 - DNA, 36 bp, Artificial Sequence
  ACA3-A5
acactctttccctacacgacgctcttccgatcttct

SEQ ID NO: 6 - DNA, 36 bp, Artificial Sequence
  ACA3-A7
gaagatcggaagagcacacgtctgaactccagtcac

SEQ ID NO: 7 - DNA, 36 bp, Artificial Sequence
  ACA4-A5
acactctttccctacacgacgctcttccgatctcat

SEQ ID NO: 8 - DNA, 36 bp, Artificial Sequence
  ACA4-A7
tgagatcggaagagcacacgtctgaactccagtcac

SEQ ID NO: 9 - DNA, 24 bp, Artificial Sequence
  Oligo PPS 1.1
acactctttccctacacgacgctc

SEQ ID NO: 10 - DNA, 23 bp, Artificial Sequence
  Oligo PPS 2.1
gtgactggagttcagacgtgtgc

(19) United States
(12) Patent Application Publication
(10) Pub. No.: US 2022/0372469 A1
(43) Pub. Date: Nov. 24, 2022

(54) PREPARATION METHOD FOR DNA LIBRARY, AND ANALYSIS METHOD FOR DNA LIBRARY

(71) Applicant: GUANGZHOU BURNING ROCK DX CO., LTD, Guangzhou, Guangdong (CN)

(72) Inventors: Zhihong ZHANG, Guangzhou (CN); Xiaotian WANG, Guangzhou (CN); Zhen ZHANG, Guangzhou (CN); Yunzhao GU, Guangzhou (CN); Yusheng HAN, Guangzhou (CN); Haibo ZHANG, Guangzhou (CN)

(21) Appl. No.: 17/620,607

(22) PCT Filed: Aug. 25, 2020
(86) PCT No.: PCT/CN2020/111054

§ 371 Date: Dec. 17, 2021

(30) Foreign Application Priority Data
Jun. 25, 2019 (CN) 201910555735.3

(51) Int. Cl. C12N 15/10; C12Q 1/6869

(52) U.S. Cl. CPC C12N 15/1065; C12Q 1/6869; C12Q 1/6806

(57) ABSTRACT

Provided is a preparation method for a DNA library, comprising a pre-library preparation process, the pre-library preparation process comprising DNA preparation, end repair and 3′ A-tailing, linker connection using an anti-contamination linker, linker connected product purification, pre-library amplification, and amplified pre-library purification. Also provided are a use of the anti-contamination linker in preparing a test kit for DNA library capture, and a method for performing bioinformatic analysis on the DNA library prepared by means of the preparation method of the present invention. The preparation method of the present invention reduces the risk of cross-contamination between samples.

### TECHNICAL FIELD

The present disclosure relates to the field of nucleic acid sequencing. Specifically, the present disclosure relates to the methods for the preparation and analysis of DNA library.

### BACKGROUND ART

With the constant discovery of gene variations closely related to drug sensitivity, drug resistance, prognosis and other clinical values, urgent clinical requirements and scientific development have promoted the reformation and innovation in the supervision mode of multi-gene detection products based on next-generation sequencing (NGS) technology in China. NGS multi-gene detection products have been greatly popularized and generalized in China in recent years, and the market demand rises rapidly. In the face of increasing amounts of samples and shortened term of detection cycles, there is greater pressure on the detection procedure of library construction of samples. Increase of operator and introduction of automated equipment may improve detection throughput, but the risk of cross-contamination of samples increase accordingly.

There is a need in the art for the methods for preparing DNA library that can reduce the risk of cross-contamination of samples.

### DESCRIPTION OF INVENTION

The present disclosure is based on the discovery of the inventors that in the last step of the procedure of DNA library construction in prior art, PCR is used to add specific tags to different samples, that is, the samples are not separated from one another until the end of the procedure, heretofore, manual experimental operation for library construction relied on physical isolation (tube cover, sealing film) to isolate different samples, and strict compliance with the experimental standard operation procedure (SOP). Increasing of operator and library construction throughput for each operator or transferring the procedure to an automated workstation in order to increase detection throughput, will virtually increase the risk of cross-contamination of samples.

In the Chinese patent application numbered 201611154433.8, which is incorporated herein by reference, the inventors provide a method for DNA library construction, on the basis of which the inventors made further improvements.

According to the present disclosure, the separation of samples is shifted to the second step—adapter ligation—from the last step of library construction, by replacing the original single adapter pair with 4 types of new adapter pairs (only 2-3 bps more than the original ones), and arranging them in different positions. Each sample is linked to one adapter pair, and 4 adapter pairs are arranged in a special pattern on the 96-well plate so that no matter how many samples there are, it can be ensured that the adapter pair used for each sample is distinct from those samples surrounding it. In combination with the updated bioinformative analysis procedure, the risk of cross-contamination can be completely eliminated.

In one aspect, the present disclosure provides a method for preparing a DNA library, which includes a pre-library preparation procedure, including DNA preparation, end repair and 3′A tailing, adapter ligation using contamination-resistant adapters, purification of adapter ligation products, amplification of pre-library and purification of the amplified pre-library, wherein contamination-resistant adapters are additionally added with 2-3 bps at the 3′- or the 5′-end compared with the original adapter used to prepare the DNA library, thus forming multiple pairs of contamination-resistant adapters.

In one example, the multiple pairs of contamination-resistant adapters are 4, 5, 6, 7 or 8 pairs.

In one example, the design of the contamination-resistant adapter pairs meets the following criteria:

(1) Add bases from the 3′-end of the original adapter, and ensure that the last base added is a T;

(2) Add A, T, G and C to the first position from the 3′-end of the original adapter to ensure signal equilibrium during sequencing and no affecting on the judgment about base detection;

(3) On each position added at the 3′-end of the original adapter, the percentage of the same base should not exceed 50%;

Following (1)-(3) above, multiple first contamination-resistant adapters are obtained;

and

(4) At the 5′-end of original adapter adding the bases that are reversely complementary to the extra bases excepting for terminal T in the first contamination-resistant adapters, and the first base at the 5′-end is phosphorylated, thus obtaining multiple second contamination-resistant adapters.

In one example, on the position of the first proximal base added at the 3′-end of the original adapter, there are 4 types of bases, each accounting for 25%; on the position of the second proximal base added at the 3′-end of the original adapter, there are 3 types of bases, with T bases accounting for 50% and the remaining 2 types of bases each accounting for 25%; on the position of the third proximal base added at the 3′- or 5′-end of the original adapter, there is no base for two adapters, and a fixed base T for the other two adapters, accounting for 50%.

In one example, the sequences of the original adapters are:

ADM-A5:
ACACTCTTTCCCTACACGACGCTCTTCCGATC*T
ADM-A7:
/5Phos/GATCGGAAGAGCACACGTCTGAACTCCAGTCAC;
*represents phosphorothioate-modification; /5Phos/ represents phosphorylation modification.

In one example, for multiple first contamination-resistant adapters, the extra base sequences are A*T, G*T, TC*T and CA*T; and for multiple second contamination-resistant adapters, the additional bases are TA, CA, GAA and TGA, * represents phosphorothioate-modification; /5Phos/ represents phosphorylation modification.

In one example, the sequences of contamination-resistant adapters are,

ACA1-A5:
ACACTCTTTCCCTACACGACGCTCTTCCGATCTA*T
ACA1-A7:
/5Phos/TAGATCGGAAGAGCACACGTCTGAACTCCAGTCAC
ACA2-A5:
ACACTCTTTCCCTACACGACGCTCTTCCGATCTG*T
ACA2-A7:
/5Phos/CAGATCGGAAGAGCACACGTCTGAACTCCAGTCAC
ACA3-A5:
ACACTCTTTCCCTACACGACGCTCTTCCGATCTTC*T
ACA3-A7:
/5Phos/GAAGATCGGAAGAGCACACGTCTGAACTCCAGTCAC
ACA4-A5:
ACACTCTTTCCCTACACGACGCTCTTCCGATCTCA*T
ACA4-A7:
/5Phos/TGAGATCGGAAGAGCACACGTCTGAACTCCAGTCAC,
which correspond to SEQ ID No. 1-8, respectively.
*represents phosphorothioate-modification; /5Phos/ represents phosphorylation modification, wherein the bases that are underlined and bolded are extra bases.

In one example, the test samples are arranged such that each of the contamination-resistant adapter is different from those at adjacent or surrounding locations.

In one example, the following primers are used for pre-library amplification:

Oligo PPS 1.1:
ACACTCTTTCCCTACACGACGCTC;
Oligo PPS 2.1:
GTGACTGGAGTTCAGACGTGTGC (corresponding to SEQ ID
No. 9-10, respectively).

In one example, the test samples are arranged such that the basic arrangement unit of the contamination-resistant adapters is:

| | |
|---|---|
| ACA1 | ACA3 |
| ACA2 | ACA4 |
| ACA3 | ACA1 |
| ACA4 | ACA2; |

wherein ACA1 means using of ACA1-A5 and ACA1-A7, ACA2 means using ACA2-A5 and ACA2-A7, ACA3 means using ACA3-A5 and ACA3-A7, and ACA4 means using ACA4-A5 and ACA4-A7.

In another aspect, the present disclosure provides the use of the adapters according to the present disclosure n in the preparation of DNA library capture kit.

In one example, the DNA library is a cfDNA library, a leukocyte gDNA library or a tissue-derived DNA library.

In another aspect, a method for bioinformative analysis of the DNA library prepared by the method according to the present disclosure is provided, which includes sequencing the DNA library and analyzing the sequencing data; if condition 1 but not condition 2 of the following two conditions is met, it is considered that a pair of reads possesses an contamination-resistant adapter at the 5′-end, and not at the 3′-end; if the following two conditions are both met, it is considered that a pair of reads possesses contamination-resistant adapters at both the 5′- and the 3′-ends.
Condition 1: Calculating Hamming distance between the primary 2-3 bps of the 5′-end of a pair of reads with same sequence ID, i.e., the read 1 sequence and the read 2 sequence respectively and the primary 2-3 bps of the 5′-end of the contamination-resistant adapter, and the sum of numerical values is less than or equal to 1;
Condition 2: In the case that condition 1 is met and a pair of reads are of equal length, the reverse complementary sequence of one read is approximately the same as the forward sequence of the other read, that is, Hamming distance calculated with the characters of sequence of the two reads is less than or equal to the default value 4 set by the software.

In one example, in the subsequent analysis procedure, for a pair of reads with contamination-resistant adapter-specific sequences merely at the 5′-end, only the 2-3 bps at the 5′-end of the read are subtracted; and for a pair of reads with contamination-resistant adapter-specific sequences at both the 5′- and the 3′-end, the 2-3 bps at both the 5′-end and 3′-end of the read are subtracted.

In one example, a pair of reads after the two types of subtractions of contamination-resistant adapters are put respectively in the fastq files of the retained read 1 and read 2 for subsequent analysis; and for a pair of reads that do not meet condition 1, it is put in the fastq files of abandoned read 1 and read 2, for subsequent inspection and analysis.

In one example, the method includes judging the type of contamination-resistant adapter, and giving the judged adapter sequence and the proportion of the type of dominant adapters during the analysis; if the proportion of the type of dominant adapters is less than 90%, it is considered that the sample has been contaminated with other samples, and subsequent analysis procedures are stopped. If the type of dominant adapter accounts for more than 90% but less than 98%, it is considered that the sample has been slightly contaminated, and the subsequent analysis procedures can be performed after removing the reads containing contaminated adapters; if the type of dominant adapter accounts for more than 98%, it is considered that the sample is not contaminated, and the subsequent analysis procedures are carried out directly.

In one example, the total number of read pairs, the number of read pairs whose adapters are cleaved, the number of read pairs eventually retained and the number of read pairs abandoned in the original data file are counted in the final results of analysis.

### DESCRIPTION OF DRAWINGS

FIG. 1: Schematic diagram of the procedure for operation of library construction in the prior art. There may be damages at the ends and breaks or cuts in the middle of fragmented cfDNAs; after treatment with the combined enzyme, the DNA is repaired, with the 3′-end added with A; short adapters without Index is ligated to both ends of the DNA (indicated by red box in dashed line) by ligase; the pre-library (whole genome) is amplified with high-fidelity enzyme; the adapters of the pre-library are blocked by blocking primers of universal short adapter (B1-B4), and the biotin (red) containing probes specific to the targeted regions hybridize to the pre-library; the pre-library bound to biotin probes is captured by streptavidin conjugated magnetic beads (blue) and eluted specifically; the eluted capture library is added with double-ended sample tags by PCR to achieve multiple sample sequencing.

FIG. 2: The procedure of library construction of the present disclosure is essentially the same as that of the prior art, except that the adapters and the primers (indicated by red box in dashed line) for pre-library amplification are replaced. There may be damages at the ends and breaks or cuts in the middle of fragmented cfDNAs; after treatment with the combined enzyme, the DNA is repaired, with the 3′-end added with A; 4 types of contamination-resistant adapters (ACA1/2/3/4) are ligated to both ends of DNA; pre-library (whole genome) is amplified utilizing PPS primers and high-fidelity enzyme; the adapters of the pre-library are blocked by blocking primers of universal short adapter (B1-B4), and biotin (red) containing probes specific to the targeted regions hybridize to the pre-library; The pre-library bound to biotin probes is captured by streptavidin conjugated magnetic beads (blue) and eluted specifically; the eluted capture library is added with double-ended sample tags by PCR to achieve multiple sample sequencing.

FIG. 3: Schematic diagram of the structure of the contamination-resistant adapter: on the position of the first base added, there are 4 types of bases, each accounting for 25%; on the position of the second base added, there are 3 types of bases, with T bases accounting for 50% and the remaining 2 types of bases each accounting for 25%; on the position of the third base added, the fixed base of ACA1 and ACA2 addition has ended, and the base on this position is the first base N of the inserted fragment, and the 4 bases are randomly distributed. ACA3 and ACA4 are still fixed bases T on this position, which accounts for 50%.

FIG. 4A-4C: Statistics of experimental QC results of library construction.

FIG. 5A-5D: Statistics of bioinformative QC results of sequencing.

FIG. 6A-6D: Statistics of experimental QC results of library construction.

FIGS. 7A-7D: Statistics of bioinformative QC results of sequencing.

FIG. 8: Detection result of EGFR A750del mutation site in the NA12878-ACA3 sample processed by the analysis procedure in the prior art.

FIG. 9: Detection result of EGFR A750del mutation site in the NA12878-ACA3 sample processed by the newly designed analysis procedure.

FIG. 10: Mind map of bioinformative analysis algorithm for contamination-resistant adapter removal.

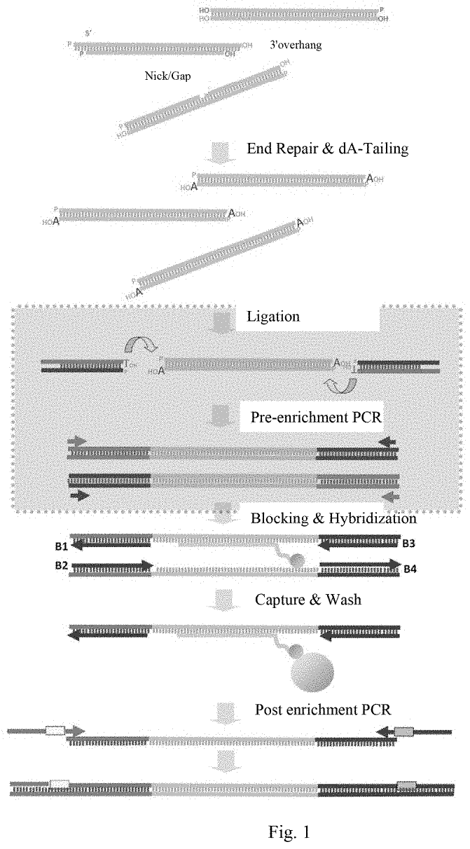

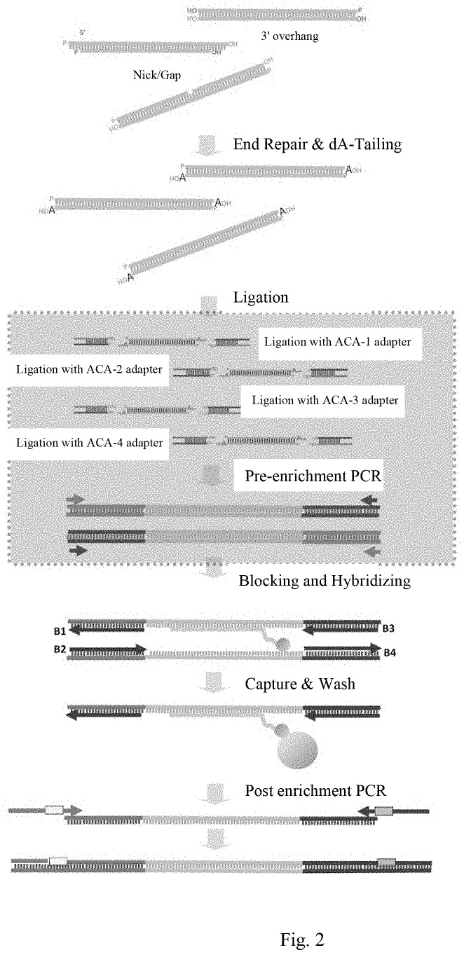

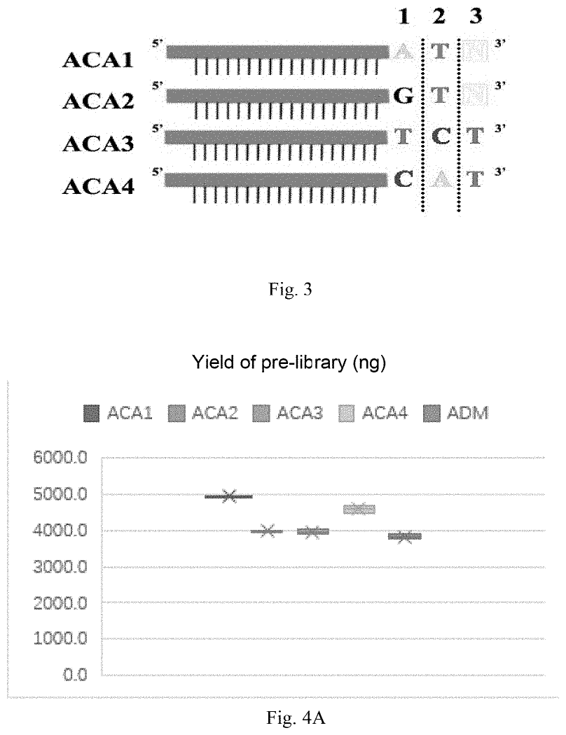

### EMBODIMENTS

The present disclosure will be further illustrated below in conjunction with specific embodiments. It should be understood that the following examples are solely used to illustrate the present disclosure and not to limit its scope of protection.

### EXAMPLES

### Methods and Materials

The experiment is carried out by using the HS library construction kit from Guangzhou Burning Rock Biotech and capture probes for detection of human multi-gene mutation (Langke). The specific operation steps are as follows.

1. Ends-Repair and 3′A-Tailing

1.1 Preparation of reagent: Open the HS library construction kit, take ERA buffer and thaw it on ice.

1.2 Setting of program: Set the PCR thermal cycler (BioRad S1000 or ABI Veriti), name the program as “ERA”, with the following conditions

Set 85° C. for lid heating, reaction volume 60 μL:
20° C., 30 minutes (note: lidded)
65° C., 30 minutes (note: lidded)
hold at 4° C.

1.3 Procedure for operation
Add nuclease-free water to a 1.5 mL tube to dilute 30 ng of sample to 50 μL. Vortex the tube and centrifuge for 3 seconds.
Transfer 50 μL sample in the 1.5 mL tube to bottoms of the wells of a 48-well plate with a single-channel pipette P100, and record and mark the order of the samples (the 48-well plate is placed on the PCR tube rack for use).
Prepare mixed solution for end repair and A-tailing reaction systems (table 1, preparing on ice) at a ratio of 1:1.1 in a new 1.5 mL Eppendorf LoBind tube.
Flick the 1.5 mL tube 3-5 times, invertit 2-3 times, centrifuge for 3 seconds.
Divide the mixed solution in aliquot between the tubes of an eight-tube strip (dispose according to the volume of sample) by using a single-channel pipette P200, avoiding formation of bubbles, and centrifuge for 3 seconds.
Take 10 μL of the mixed solution from eight-tube strip by using an eight-channel pipette P10 into a 48-well plate (50 μL sample already included), pipette 10 times, stick a film (micro seal B) on and make it fit tightly with the plate by using a scrape. Make sure that there is no bubble in the tube. Centrifuge at 1000 rpm for 3 seconds.

TABLE 1
End repair and 3′A-tailing

| | Volume per |
|---|---|
| Reagents | reaction |
| End Repair and A-Tailing buffer | 7 μL |
| Enzyme mixture solution for End Repair and A-Tailing | 3 μL |
| Fragmented DNA | 50 μL |
| Total | 60 μL |

Put the 48-well plate in the PCR thermal cycler Bio-Rad S1000 or ABI Veriti, using the program “ERA” (lid heating at 85° C., 30 minutes at 20° C., 30 minutes at 65° C., hold at 4° C.). Go to the next step within 2 hours.

2. Adapter Ligation

2.1 Preparation of reagent: prepare the reagents in Table 2.

TABLE 2
Adapter ligation and reagent purification

| Reagents | Preparation |
|---|---|
| ligation buffer | Thaw on ice |
| DNA ligase | On ice |
| ACA adapter | Thaw on ice |
| SPB | Equilibrate at RT (RT) for 30 minutes |
| Ethanol | RT (for purification) |

2.2 Setting of program: Set BioRad S1000 or ABI Veriti, name the program as “LIG”.

Set 85° C. for lid heating, reaction volume 50/100 μl:
20° C., 15 minutes (note: not lidded)
70° C., 10 minutes (note: lidded)
hold at 4° C.

2.3 Operation procedure
Take out the 48-well plate that has completed the reaction program “ERA” from the PCR thermal cycler, place it on PCR tube rack, centrifuge at 1000 rpm for 3 seconds. Tear off the sealing film (micro seal B) carefully and keep it on ice for use.
Prepare mixed solution for adapter ligation reaction system (Table 3) at a ratio of 1:1.1 in a 1.5 mL Eppendorf LoBind tube on ice.
Flick the 1.5 mL tube 3-5 times, invert it 2-3 times, and centrifuge for 3 seconds.
Take the corresponding mixed solution into an eight-tube strip by using a single-channel pipette P200, and centrifuge for 3 seconds.
Take 50 μL of the mixed solution from the eight-tube strip into the above 48-well plate by using an eight-channel pipette P200, whose volume scale is adjusted to 80 μL, pipette the solution gently 10 times, stick a film (micro seal B) on and make it fit tightly with the plate by using a scrape. Make sure that there is no bubble in the tube. Centrifuge at 1000 rpm for 3 seconds.

TABLE 3
Set of ligation reaction

| Reagents | Volume per reaction |
|---|---|
| Ligation buffer | 30 μL |
| DNA ligase | 10 μL |
| ACA adapter | 10 μL |
| End repair mixture solution | 60 μL |
| Total volume | 110 μL |

Put the 48-well plate in the PCR thermal cycler (S1000 or ABI Veriti), run the program “LIG”
   20° C., 15 minutes (85° C. for lid heating, not lidded)
   70° C., 10 minutes, hold at 4° C. (85° C. for lid heating, lidded)

3. Purification of the Adapter Ligation Products (Ligation Purification)

3.1 Preparation
Leave the SPB magnetic beads at room temperature (“RT”) for at least 30 minutes.
Prepare fresh 75% ethanol, 400 μL for each library.

3.2 Operation procedure
Invert the tubes with SPB magnetic beads 2-3 times, vortex for 5-10 s at the maximum speed of VORTEX to homogenize.
Transfer the corresponding SPB magnetic beads into sample-loading slots by using a single-channel pipette P1000. Each sample requires 88 μL SPB magnetic beads (sample: magnetic beads=1:0.8).
Take out the 48-well plate from PCR thermal cycler, place it on the PCR tube rack, centrifuge at 1000 rpm for 3 seconds, and tear off the film carefully. Transfer 88 μL SPB magnetic beads (sample: magnetic beads=1:0.8) from sample-loading slot by using a eight-channel pipette P200 into the 48-well plate. The volume scale of the eight-channel pipette P200 is adjusted to 180 μL, and pipette the mixture 10 times.
Stick a film (micro seal B) on the 48-well plate, centrifuge at 1000 rpm for 3 s. Leave it at RT for 10 minutes.
Centrifuge at 1000 rpm for 1 min, discard the film.
Place the 48-well plate on a magnetic stand (Thermo Scientific, AM10027) and let the solution clarify (about 3-5 min).
Discard the supernatant carefully by using an eight-channel pipette P200, with the volume scale adjusted to the maximum. Note: Do not touch the magnetic beads.
With the 48-well plate still on the magnetic stand, add 200 μL of freshly prepared 75% ethanol into the sample well (in the sample-loading slot) by using an eight-channel pipette P200.
Move horizontally the 48-well plate back and forth on the magnetic stand to fully soak the magnetic beads, leave it for 1 min and discard the ethanol.
Repeat the above two steps once.
Allow the 48-well plate to sit on the magnetic stand for 1 min, and remove residual ethanol by using an eight-channel pipette P20.
Remove the 48-well plate from the magnetic stand and place it on the PCR plate rack at RT for 2 minutes to dry the magnetic beads, until that the surface of the magnetic beads is not reflective and free of cracks.
Add an appropriate amount of EB eluant into sample-loading slot.
Add 28 μL of EB solution to the 48-well plate by using an eight-channel pipette P200, cap the eight-tube strip, vortex for about 5 seconds, and centrifuge at 1000 rpm for 3 seconds.
Incubate the 48-well plate at RT for 2 minutes.
Centrifuge the plate at 1000 rpm for 1 min.
Take off the cap of eight-tube strip carefully, and place the 48-well plate on the magnetic stand for 2 minutes until the solution is clear.
Transfer 27.5 μL of the supernatant to a new 48-well plate by using an eight-channel pipette P20, aspirate all the supernatant as much as possible.

4. Amplification of Pre-Library

4.1 Preparation of reagents: See table 4.

TABLE 4
Preparation of PCR and PCR product purification

| Reagents | Preparation |
|---|---|
| 5x HiFi buffer | thaw on ice |
| 10 mM dNTP mixture | thaw on ice |
| PPS primers | thaw on ice |
| HiFi HotStart | on ice |
| SPB | equilibrate at RT for 30 minutes |
| Ethanol | RT (for purification) |

4.2 Setting of program:

“PRE” is as in table 5:

TABLE 5
Set of pre-enrichment PCR

| Step | Cycle | Temperature | Time | |
|---|---|---|---|---|
| 1 | 1 | 98° C. | 45 | s |
| 2 | 9 | 98° C. | 15 | s |
| | | 60° C. | 30 | s |
| | | 72° C. | 30 | s |
| 3 | 1 | 72° C. | 2 | min |

| | | | |
|---|---|---|---|
| 4 | 1 | 4° C. | hold |

4.3 Operation procedure
Prepare the mixed solution of reaction system (preparing on ice) according to table 6, flick 3-5 times, invert 2-3 times, centrifuge for 3 seconds, and divide in aliquot between the tubes of an eight-tube strip.
Add 22.5 μL of the mixed solution to each well (containing 27.5 μL of purified product) of the 48-well plate used in the step of “ligation product purification” by using an eight-channel pipette P200, and adjust the volume scale of the eight-channel pipette P200 to 40 μL and pipette gently 10 times.
Stick a film (micro seal B) with a scraper until it fits tightly, without bubble forming in the tube, and centrifuge at 1000 rpm for 3 s.
Place the plate in the PCR thermal cycler and run program “PRE”

TABLE 6
Pre-Enrichment PCR system

| Reagents | Volume per reation | |
|---|---|---|
| HiFi Fidelity buffer (5X) | 10 | μL |
| 10 mM dNTP Mix | 1.5 | μL |
| PPS primer | 10 | μL |
| 1 U/μL HiFi HotStart (100 U) | 1 | μL |
| Cleared Ligation Mix | 27.5 | μL |
| Total volume | 50 | μL |

5. Purification of Amplified Pre-Library

5.1 Preparation of reagents
Keep the magnetic beads at RT for at least 30 minutes.
Prepare fresh 75% ethanol, 400 μL for each library.

5.2 Operation procedure
Invert the tube of SPB magnetic beads 2-3 times, and mix them for 5-10 s at the maximum speed of VORTEX to homogenize them.
Transfer the corresponding SPB magnetic beads into the sample-loading slots by using the single-channel pipette P1000. 60 μL of SPB magnetic beads is added to each sample (sample: magnetic beads=1:1.2).
Take out the 48-well plate from the PCR thermal cycler, centrifuge at 1000 rpm for 3 seconds, and tear off the film carefully. transfer 60 μL of SPB magnetic beads (sample: magnetic beads=1:1.2) from the sample-loading slot and add them into the 48-well plate by using an eight-channel pipette P200. Adjust the volume scale of the eight-channel pipette P200 to 80 μL, and pipette the mixture gently 10 times.
Stick a film on the 48-well plate. Leave it at RT for 10 min.
Centrifuge at 1000 rpm for 1 min.
Discard the film, place the 48-well plate on the magnetic stand, and let the solution clarify (about 3-5 min).
Discard the supernatant carefully by using an eight-channel pipette P200 adjusted to its maximum volume scale of. Do not touch the magnetic beads.
With the 48-well plate still placed on the magnetic stand, add 200 μL of freshly prepared 75% ethanol to the sample-loading slot by using an eight-channel pipette P200.
Move horizontally the 48-well plate back and forth on the magnetic stand to fully soak the magnetic beads. Leave it for 1 min and discard the ethanol.
Repeat the above two steps once.
Allow the 48-well plate to sit on the magnetic stand for 1 min, and remove residual ethanol by using an eight-channel pipette P20.
Take off the 48-well plate from the magnetic rack and place it on the PCR plate rack at RT for 2 minutes to dry the magnetic beads, until that the surface of the magnetic beads is not reflective and free of cracks.
Add an appropriate amount of nuclease-free water to the sample-loading slot (EB cannot be used in this step).
Add 16 μL of nuclease-free water to the 48-well plate by using an eight-channel pipette P200, cap the eight-tube strip, vortex for about 5 seconds, and centrifuge at 1000 rpm for 3 seconds.
Incubate the 48-well plate at RT for 2 minutes.
Centrifuge at 1000 rpm for 1 min.
Discard the film and place the 48-well plate on the magnetic stand for 2 minutes until the solution is clear.
Transfer 15.5 μL of the supernatant to a new 48-well plate by using an eight-channel pipette P10. Do not aspirate magnetic beads.

6. Quality Control for the Purified Pre-Library (Pre-Library QC)
Dilution of pre-library

Take 1 μL of the purified pre-library into a new 48-well plate, add 11 μL ddH₂O, pipette using a P20 pipette 10 times to homogenize (1 μL is used for Qubit quantification, 10 μL for the next Labchip or 2100 QC)

7. Hybridization of Pre-Library (Pre-Library Hybridization)

7.1 Preparation of reagents:

Prepare the reagents in table 7.

7.2 Setting of program:

Set BioRad S1000, name the program as “HYB”
95° C. 5 min (lid heating at 105° C.)
Hold at 65° C.

TABLE 7
Preparation of hybridization reagents

| Reagent | Preparation |
|---|---|
| HYB buffer | thaw at RT |
| BLM blocking agent | thaw on ice |
| RIB blocking agent | thaw on ice |
| Langke probes | thaw on ice |

7.3 Operation procedure
Take 15 μL of the pre-library library, place it in a 48-well plate according to the maker, add 4 μl of BLM blocking agent (marked as component A) to each well, mix by pipetting 8-10 times, and cap the eight-tube strip.
Prepare component B on ice according to table 8, and then dispense it into a new eight-tube strip and cap the eight-tube strip.

### Table 8: Proportion of Component B System

| Component B | Volume per reaction | |
|---|---|---|
| HYB blocking agent | 10 | μL |
| RIB blocking agent | 0.5 | μL |
| Langke probe | 0.5 | μL |
| Total volume | 11 | μL |

component A in the PCR thermal cycler, and run the program “HYB” (95° C., 5 min; 65° C., hold).
Let the temperature of the PCR thermal cycler drop to 65° C., then place component B in the PCR thermal cycler and incubate, and close the heated lid.
After 2 minutes, open the heated lid of the PCR thermal cycler and the cap of the eight-tube strip, transfer component B to component A quickly using a pipette, replace the pipette tip each time, pipette 5 times to homogenize (keep the 48-well plate in the PCR thermal cycler), cap the eight-tube strip tightly and stick a film to prevent evaporation, close the lid of thermal cycler, and incubate at 65° C. for 16-24 h (lid heating at 105° C.).

8. Capture and Elution (Binding and Wash)

8.1 Preparation of reagents

TABLE 9
Preparation of reagents for capturing SCB magnetic beads

| Reagent | Preparation |
|---|---|
| BWS binding buffer | RT |
| Washing buffer 1 | RT |
| Washing buffer 2 | RT |
| SCB (T1 magnetic beads) | equilibrate at RT for 30 min |

Prepare the reagents in table 9.
Set temperature of thermostatic metal bath to 65° C.
Equilibrate the SCB magnetic beads at RT for more than 30 minutes.

8.2 Setting of program:

Set BioRad S1000 or ABI, and name the program as “WASH 2”:
Hold at 65° C. (Lid heating at 70° C.)

8.3 Operation procedure
Place 600 μL of WB solution 2 per sample in a 15 mL conical tube, incubate on a metal heater at 65° C.
Take out SCB/T1 magnetic beads, invert the tube 5 times to homogenize, vortex for 10 seconds, allow it to sit at RT for more than half an hour, vortex for 10 seconds, put into 1.5 ml LoBind tube according to numbers of samples, each sample requires 25 μL, with 150 μL at maximum per Lobind tube. Allow the tubes to sit on a 16-well magnetic stand for 3 minutes, and discard the supernatant.
Add 150 μL of BWS binding buffer to each 25 μL of original magnetic beads, vortex for 3 seconds, centrifuge briefly, allow the mixture to sit on the magnetic stand for 3 minutes, and discard the supernatant.
Repeat the above steps 2 times, for a total of 3 times.
Resuspend SCB (add 150 μL BWS binding buffer to each 25 μL original magnetic beads), add it to the sample-loading slot, dispense 150 μL/tube into a 48-well plate (containing 28 μL hybridization buffer) with multi-channel pipette, pipette gently 10 times, then stick a film and centrifuge at 1000 rpm for 3 seconds.
Place the plate on a thermomixer and incubate at RT 300 rpm for 30 min.
Centrifuge at 1000 rpm for 1 min, discard the film, then place the plate on a magnetic stand for 5 min, and discard the supernatant.
Add an appropriate amount of WB solution 1 into the sample-loading slot, add 150 μL of WB solution 1 to each sample well by using a P200 pipette, and adjust the volume scale of pipette to 140 μL and pipette 10 times. Stick a film. Place on a super thermomixer and incubate at RT 300 rpm for 15 min.
During the incubation process, transfer manually 2 μL of the positive controls in wells adjacent to the negative ones in wells G1, H2 and G3 to the negative controls, so as to mimic cross-contamination of samples in the experiment.
Centrifuge at 1000 rpm for 1 min, discard the film, allow the plate to sit on a magnetic stand for 5 minutes, aspirate and discard the supernatant with an eight-channel pipette P200, and then aspirate the remaining liquid with an eight-channel pipette P20.
Add an appropriate amount of WB solution 2 that has been preheated to 65° C. into the sample-loading slot. Add 150 μL of WB solution 2 to each sample well by using a P200 pipette, adjust the volume scale of the pipette to 130 μL and pipette gently 10 times to homogenize. Stick a film and place the plate in the PCR thermal cycler and incubate at 65° C. for 10 min (note: 70° C., lidded).
Centrifuge at 1000 rpm for 1 min, tear off the film, place the plate on a magnetic stand, aspirate and discard the supernatant with an eight-channel pipette P200, and then aspirate the remaining liquid with an eight-channel pipette P20.
Repeat the operation with WB solution 2 three times, for a total of four times.
Centrifuge at 1000 rpm for 1 min, place the plate on a magnetic stand, and aspirate the remaining liquid by using a P20 pipette.
Add an appropriate amount of EB into the sample-loading slot.
Add 20 μL of EB to the sample well, cap the eight-strip tube, vortex for 3 seconds, resuspend the SCB magnetic beads, and centrifuge at 1000 rpm for 3 seconds.

9. Preparation of the Post Library (Post Capture Library Amplification)

9.1 Preparation of reagents: prepare the reagents in table 10.

TABLE 10
Amplification and purification of capture library

| Reagent | Preparation |
|---|---|
| 2X HiFI ready mix | thaw on ice |
| SetA/SetB/SetC/SetD series | thaw on ice |
| SPB | equilibrate at RM for 30 min |
| Ethanol | RT (for purification) |

9.2 Setting of program: set PCR thermal cycler (BioRad 51000) program as “POST” according to table 11.

TABLE 11
Post PCR setting

| Step | Cycle | Temperature | Time |
|---|---|---|---|
| 1 | 1 | 98° C. | 45 s |
| | | 98° C. | 15 s |
| 2 | 14 | 60° C. | 30 s |
| | | 72° C. | 30 s |
| 3 | 1 | 72° C. | 10 min |
| 4 | 1 | 4° C. | hold |

9.3 Operation procedure
Take a new 48-well plate and add HiFi ready mix and Index according to the PostPCR system shown in Table 12. The concrete operations are as follows:

a. Put the HIFI ready mix and Index on the ice to thaw, prepare a new 48-well plate and sort the thawed Index.

b. Add 5 μl Index to the wall of tube of the corresponding well by using a single-channel pipette P2.5, cap the eight-tube strip, and after confirming that all of them have been added, centrifuge the plate at 1000 rpm for 3 seconds.

c. Prepare a new eight-tube strip and add an appropriate amount of HIFI readymix.

d. Aspirate 25 μl of HIFI readymix from the eight-tube strip by using a P200 pipette and add it to the 48-well plate.
Aspirate carefully 20 μl of SCB-bound magnetic beads in the 48-well plate resulted from the end of step 7.3 into the 48-well plate with Index and Mix added by using the eight-channel pipette P200, and pipette gently 10 times to homogenize. Stick a film.
Run the “POST” program on the PCR thermal cycler.

TABLE 12
Post library system

| Reagent | Volume per reaction |
|---|---|
| Captured Library with SCB | 20 μL |
| HiFi HotStart readyMix (2X) | 25 μL |
| Index | 5 μL |
| Total volume | 50 μl |

10. Purification of Post Library (Post PCR Library Purification)

10.1 preparation of the experiment
Leave the SPB magnetic beads at RT for at least 30 minutes.
Prepare fresh 75% ethanol, 400 μL for each library.

10.2 Operation procedure
Invert the tube containing the SPB magnetic beads 2-3 times, and mix them for 5-10 s at the maximum speed of VORTEX to homogenize.
Take out the PCR products (including SCB magnetic beads) from the PCR thermal cycler and centrifuge at 1000 rpm for 1 min, and allow them to sit on the magnetic stand for 5 min. Aspirate 50 μL of the supernatant and add it to a new 48-well plate by using an eight-channel pipette P20.
Aspirate corresponding SPB magnetic beads and add into the sample-loading slot using a single-channel pipette P1000. Add 50 μL SPB magnetic beads to each sample (sample: magnetic beads=1:1).
Aspirate 50 μL of SPB magnetic beads (sample: magnetic beads=1:1) from the sample-loading slot and add them to a 48-well plate (containing 50 μL of PCR product with SCB beads removed) by using an eight-channel pipette P200. Adjust the volume scale of the eight-channel pipette P200 to 80 μL, and pipette the beads gently 10 times.
stick a film on the 48-well plate. Leave it at RT for 10 minutes.
Centrifuge at 1000 rpm for 1 min.
Discard the film, place the 48-well plate on the magnetic stand, and let the solution clarify (about 3-5 min).
Adjust the volume scale of the eight-channel pipette P200 to the maximum, and discard the supernatant. Do not touch the magnetic beads.
The 48-well plate is still on the magnetic stand. Add 200 μL of freshly prepared 75% ethanol to the sample-loading slot by using an eight-channel pipette P200.
Move horizontally the 48-well plate back and forth on the magnetic stand to fully soak the magnetic beads. Wait for 1 min and discard the ethanol.
Repeat the above two steps once.
Allow the 48-well plate to sit on the magnetic stand for 1 min, remove residual ethanol by using an eight-channel pipette P20.
Remove the 48-well plate from the magnetic rack and place it on the PCR plate rack at RT for 2 minutes to dry the magnetic beads, until that the surface of the magnetic beads is not reflective and free of cracks.
Add an appropriate amount of EB into the sample-loading slot.
Add 20 μL of EB to the 48-well plate by using an eight-channel pipette P20, cap the eight-tube strip, vortex for about 5 seconds, and centrifuge at 1000 rpm for 3 seconds.
Incubate the 48-well plate at RT for 2 minutes.
Centrifuge at 1000 rpm for 1 minute.
Discard carefully the cover of the eight-tube strip, and place the 48-well plate on the magnetic stand for 2 minutes until the solution is clear.
Transfer 19.5 μL of the supernatant to a new 48-well plate using an eight-channel pipette P10. Do not aspirate magnetic beads.

11. Detection of Concentration of the Purified Library (Library QC)
Take 2 μL of the purified library in a new 1.5 mL EP tube, add 10 μL ddH₂O (1 μL is used for Qubit quantification, and the remaining 11 μL is used for the next step for Labchip or 2100 QC)

12. Detection of Fragment Size of Purified Library (Library QC)

12.1 Preparation of reagent
Allow Labchip HS or Agilent 2100 HS reagents and chips to sit at RT for more than 30 minutes.

12.2 Experimental procedure
Detection reagent: Labchip HS Kit
Detection method: Use the 10 μL library in step 3.14.3 for the detection in the present step (refer to “Standard Operation Procedure for Labchip Detection” or “Standard Operation Procedure for Agilent 2100 HS DNA Kit”).

### Procedure of Bioinformative Analysis:

In the course of the library construction of samples according to the present disclosure, new contamination-resistant adapters and special arrangement pattern are introduced in order to ensure that each sample carries a specific tag in the early stage of the experiment. Even if cross-contamination occurs in the later stage of the experiment, it can be detected in the bioinformative analysis procedure and the information from external contamination can be rejected, reduced, and the risk of pollution can even be eliminated.

Check each pair of reads of off-line fastq file, and output the result of cross-contamination statistics at the same time, thus ensuring the accuracy of the data used in subsequent analysis steps; and in the course of analysis of the contamination-resistant adapters, the reads with contamination-resistant adapters will be cleaved, and the reads with contamination-resistant adapters are re-input into a new file. In this way it is easy for subsequent search and verification.

The innovation of the present disclosure reside in that the software will judge automatically the type of contamination-resistant adapters in off-line fastq file, and then execute the subsequent analysis procedure or judge the contamination sources of the sample from the type of adapters contaminated, which simplifies the operation procedure during the running of software.

### The Realization Principle of the Present Disclosure Includes Design, Experimental Method and Algorithm.

A file in bcl format is generated after the sequencing data is offlined, and then the file in bcl format is converted into a file in fastq format by bcl2fastq software, however, the bcl2fastq software will execute forced pruning of the sequencing reads, that is, as long as the preset adapter appears at the 3′-end of the read sequence, the bases present at the 5′-end will be pruned, so the reads off-lined are not of equal length, and their lengths are less than or equal to the number of cycles that the sequencer run; but if some of insert sequences are too short while the library is constructed, and their lengths are less than that of the read being sequenced, adapter sequences will be detected at the 3′-end after the reads sequence is generated. Therefore, the method for removing the contamination-resistant adapters has been designed in the light of above characteristics.

The contamination-resistant adapters designed by the present kit are all modified adapters (ADM) from original kit, that is, add 2-3 bps to the 5′-end of ADM (A7), and add to the 3′-end of ADM (A5) the bases of the same length that are reversely complementary to the 5′-end. Therefore, after the sequencing data is off-lined, if the first but not the second of the following two conditions is met, it is considered that this pair of reads possesses a contamination-resistant adapter at the 5′-end but not at the 3′-end. If the following two conditions are both met, it is considered that this pair of reads possesses contamination-resistant adapter at both the 5′-end and the 3′-end.
Condition 1: calculating Hamming distance between the primary 2-3 bps of the 5′-end of a pair of reads with same sequence ID, i.e., the read 1 sequence and the read 2 sequence respectively and the 2-3 bps of the 5′-end of the contamination-resistant adapter (A7), and the sum the numerical values is less than or equal to 1;
Condition 2: In the case that condition 1 is met, and a pair of reads are of equal length, the reverse complementary sequence of one read is approximately the same as the forward sequence of the other read (It is considered as approximately the same if Hamming distance calculated with the sequence characters of the two reads is less than or equal to the default value 4 set by the software).

In the process of subsequent analysis, for a pair of reads with contamination-resistant adapter-specific sequences merely at the 5′-end, only the 2-3 bps at the 5′-end of the read are subtracted; and for a pair of reads with contamination-resistant adapter-specific sequences at both the 5′- and the 3′-end, the 2-3 bps at both ends are subtracted. Put a pair of reads after the above two types of subtraction of contamination-resistant adapter specific sequences in the fastq files of retained read 1 and read 2; and for a pair of reads that do not meet condition 1, they are put in the fastq files of abandoned read 1 and read 2, for subsequent inspection and analysis.

The software will judge the type of contamination-resistant adapter of off-line raw fastq file, and giving the proportion of the judged adapter sequences and the type of dominant adapter during the analysis; if the proportion of type of the dominant adapter is less than 90%, it is considered that the sample has been contaminated with other samples, and the subsequent analysis procedures are stopped. If the dominant adapter type accounts for more than 90% but less than 98%, it is considered that the sample has been slightly contaminated with other samples, and the subsequent analysis procedures can be performed after removing the reads containing the contaminated adapter; If the dominant adapter type accounts for more than 98%, it is considered that the sample are not contaminated, and the subsequent analysis procedures are carried out directly. The total number of read pairs of the original data file, the number of read pairs whose adapter are cleaved, the number of read pairs eventually retained and the number of abandoned read pairs are counted in the final results of analysis.

Adapters and Primers

ADM adapters are as follow:

ADM-A5:
ACACTCTTTCCCTACACGACGCTCTTCCGATC*T
ADM-A7:
/5Phos/GATCGGAAGAGCACACGTCTGAACTCCAGTCAC;

ACA1 [symbol] ACA2 [symbol] ACA3 and ACA4 adapters are as follow:

ACA1-A5:
ACACTCTTTCCCTACACGACGCTCTTCCGATCTA*T
ACA1-A7:
/5Phos/TAGATCGGAAGAGCACACGTCTGAACTCCAGTCAC
ACA2-A5:
ACACTCTTTCCCTACACGACGCTCTTCCGATCTG*T
ACA2-A7:
/5Phos/CAGATCGGAAGAGCACACGTCTGAACTCCAGTCAC
ACA3-A5:
ACACTCTTTCCCTACACGACGCTCTTCCGATCTTC*T
ACA3-A7:
/5Phos/GAAGATCGGAAGAGCACACGTCTGAACTCCAGTCAC
ACA4-A5:
ACACTCTTTCCCTACACGACGCTCTTCCGATCTCA*T
ACA4-A7:
/5Phos/TGAGATCGGAAGAGCACACGTCTGAACTCCAGTCAC

Optimized PPS primers (PPO Plus primers) according to the present application:

Oligo PPS 1.1:
ACACTCTTTCCCTACACGACGCTC;
Oligo PPS 2.1:
GTGACTGGAGTTCAGACGTGTGC.

Blocking Primers:

PCR1B1
ACACTCTTTCCCTACACGACGCTCTTCCGATCT
PCR1B2
AGATCGGAAGAGCGTCGTGTAGGGAAAGAGTGT
PCR2B1
GTGACTGGAGTTCAGACGTGTGCTCTTCCGATC
PCR2B2
GATCGGAAGAGCACACGTCTGAACTCCAGTCAC.

### Example 1: Verification of the Efficiency of Library Construction of Contamination-Resistant ACA Adapter and PPO Plus Primers in the Experiment

NA12878 genomic DNA (Coriell Institute, catalog number NA12878) (negative control) was taken as the experimental sample, and 30 ng input amount after 195 s interruption by Covaris M220 interrupter was used to hybridization capture for library construction. The newly designed ACA adapter and PPS primers were employed in the process of library construction, and ADM adapter and PPO primers (sequence (5′→3′): ACACTCTTTCCCTACACGACG; GTGACTGGAGTTCAGACGTG) were used as experimental controls to verify the efficiency of newly designed adapter primer for library construction. The arrangement of ACA and ADM adapters is shown in table 13 below. The process of DNA library preparation is as described above.

TABLE 13
Schematic diagram of arrangement
patterns of experimental adapters

| | 1 | 2 |
|---|---|---|
| A | ACA1 | ACA1 |
| B | ACA2 | ACA2 |
| C | ACA3 | ACA3 |
| D | ACA4 | ACA4 |
| E | ADM | ADM |

### Experimental Results

1) QC of Library Construction

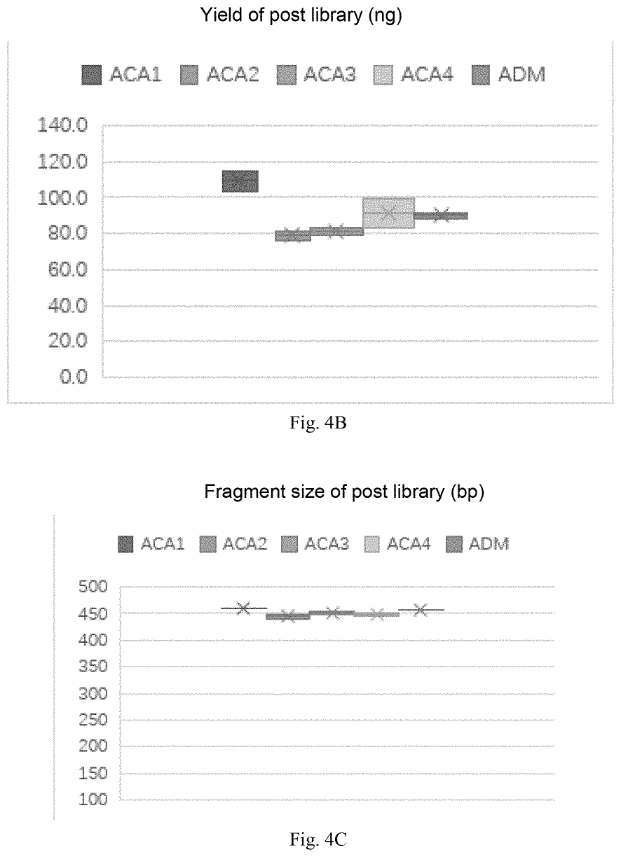

The QC index of library construction in this example focuses on yield of pre-library, yield of post library, and average fragment size of the post library. For the detailed experimental QC results and statistics, see table 14 below, and the statistical information is shown in FIGS. 4A-4C.

TABLE 14
QC of library construction

| | | | | Fragment |
|---|---|---|---|---|
| | Well | Yield of pre- | Yield of final- | size of final- |
| Sample name | position | library(ng) | library(ng) | library(bp) |
| NA12878-ACA1 | A1 | 4893.7 | 103.9 | 459 |
| NA12878-ACA1 | A2 | 4972.8 | 114.5 | 461 |
| NA12878-ACA2 | B1 | 3949.2 | 76.2 | 449 |
| NA12878-ACA2 | B2 | 4006.1 | 81.2 | 440 |
| NA12878-ACA3 | C1 | 3889.0 | 79.0 | 448 |
| NA12878-ACA3 | C2 | 4041.8 | 83.4 | 454 |
| NA12878-ACA4 | D1 | 4707.9 | 99.5 | 450 |
| NA12878-ACA4 | D2 | 4491.4 | 83.0 | 446 |
| NA12878-ADM | E1 | 3907.2 | 91.4 | 456 |
| NA12878-ADM | E2 | 3751.9 | 88.4 | 457 |

The results show that the newly designed ACA adapter primers and PPO Plus primers both meet or exceed the criteria for library construction in terms of pre-library yield compared with original ADM adapter and PPO primers. With the corresponding 1 μg of pre-library input for hybridization capture, yield of post library and average fragment size obtained in the experimental group are similar to those in the control group.

This demonstrates that the newly designed ACA adapter and PPS primers can meet QC index of normal library construction, and to a certain extent, is better in terms of efficiency of library construction than with original ADM adapter and PPO primers.

2) Bioinformative QC of Sequencing

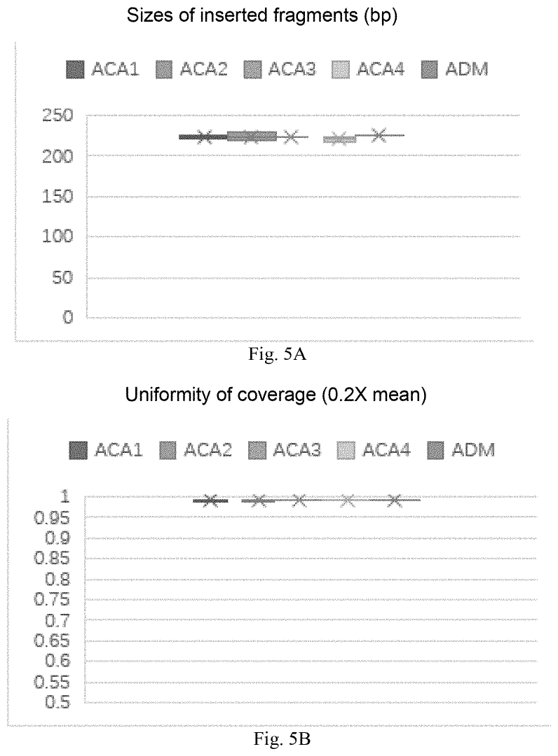

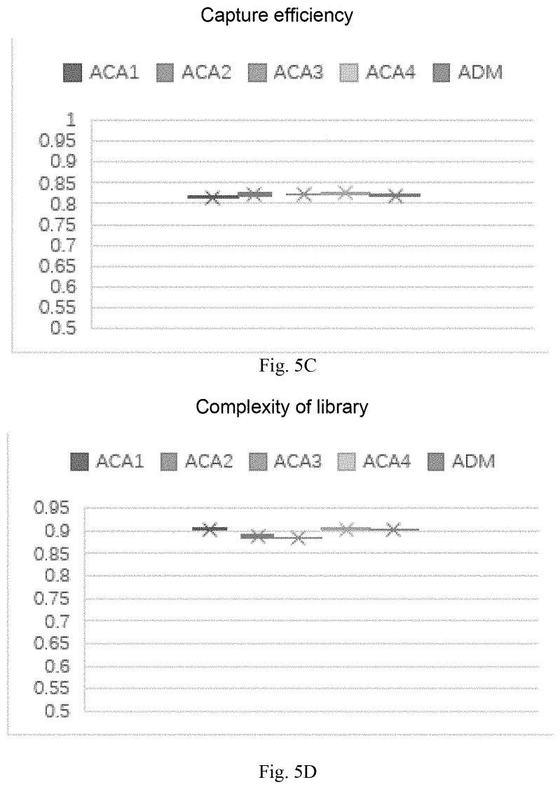

The QC index of targeted capture experiment in this example mainly focuses on size of inserted fragment, capture efficiency, complexity of library construction and uniformity of coverage (0.2× mean). For the detailed QC results, see table below. The statistical information is shown in FIGS. 5A-5D.

TABLE 15
Bioinformative QC of sequencing

| | | Sizes of | complexity | | Unifor- |
|---|---|---|---|---|---|
| | | inserted | of library | | mity of |
| Sample | Well | fragments | construction | Capture | coverage |
| name | position | (bp) | (ng) | efficiency | (0.2X mean) |
| NA12878- | A1 | 225 | 0.902 | 0.815 | 0.991 |
| ACA1 | | | | | |
| NA12878- | A2 | 222 | 0.905 | 0.817 | 0.99 |
| ACA1 | | | | | |
| NA12878- | B1 | 229 | 0.886 | 0.817 | 0.991 |
| ACA2 | | | | | |
| NA12878- | B2 | 219 | 0.893 | 0.826 | 0.99 |
| ACA2 | | | | | |
| NA12878- | C1 | 223 | 0.885 | 0.823 | 0.991 |
| ACA3 | | | | | |
| NA12878- | C2 | 223 | 0.886 | 0.821 | 0.991 |
| ACA3 | | | | | |
| NA12878- | D1 | 223 | 0.902 | 0.824 | 0.991 |
| ACA4 | | | | | |
| NA12878- | D2 | 218 | 0.905 | 0.828 | 0.991 |
| ACA4 | | | | | |
| NA12878- | E1 | 226 | 0.903 | 0.819 | 0.991 |
| ADM | | | | | |
| NA12878- | E2 | 226 | 0.901 | 0.822 | 0.991 |
| ADM | | | | | |

The results showed that the newly designed ACA adapter and PPO Plus primers had no significant difference in terms of sizes of inserted fragments, capture efficiency, complexity of library construction and uniformity of coverage (0.2× mean) compared with the original ADM adapter and PPO primers.

This demonstrates that the newly designed ACA adapter and PPO Plus primers can meet the requirements for QC analysis of normal sequencing and has the same effect of analysis as with the original ADM adapter and PPO primers.

### Example 2: Verification of the Ability of Adapters of Present Disclosure to Resist Contamination During Experiment

### Experimental Design

NA12878 genomic DNA served as negative control, and DNA from HCC827 cell line (HCC827 cell line was purchased from ATCC, and DNA was extracted with an extraction kit from Tiangen, Item No. DP304) was used as positive control for the experiment (information about HCC827 cell line mutation: EGFR E746-A750 del, AF=83.4%, EGFR CNV=37), both samples were used in hybridization capture of library construction in a 30 ng input amount after 195 s interruption by Covaris M220 interrupter. The samples were arranged according to the checkerboard method, and the specific arrangement is shown in Table 16.

TABLE 16
Schematic diagram of arrangement pattern of experimental samples

| | 1 | 2 | 3 | 4 |
|---|---|---|---|---|
| A | N | H | N | H |
| B | H | N | H | N |
| C | N | H | N | H |
| D | H | N | H | N |
| E | N | H | N | H |
| F | H | N | H | N |
| G | N | H | N | H |
| H | H | N | H | N |

Notes:
N represents NA12878 (negative control);
H represents HCC827 (positive control)

The newly designed ACA adapter was employed in the process of library construction, and the original ADM adapter was used as an experimental control (see table 17 for the specific arrangement of the adapters). After the pre-library was completed, 1 μg input amount of the pre-library was taken respectively for hybridization capture, and the positive controls in wells adjacent to the negative controls were introduced manually to the negative controls in wells G1, H2 and G3 to mimic cross-contamination of samples, thus verifying the ability of newly designed ACA adapter to resist contamination.

TABLE 17
Schematic diagram of arrangement pattern of experimental adapters

| | 1 | 2 | 3 | 4 |
|---|---|---|---|---|
| A | ACA1 | ACA3 | ACA1 | ADM |
| B | ADA2 | ACA4 | ACA2 | ADM |
| C | ACA3 | ACA1 | ACA3 | ADM |
| D | ACA4 | ACA2 | ACA4 | ADM |
| E | ACA1 | ACA3 | ACA1 | ADM |
| F | ACA2 | ACA4 | ACA2 | ADM |
| G | ACA3 | ACA1 | ACA3 | ADM |
| H | ACA4 | ACA2 | ACA4 | ADM |

Experimental Results:

1) QC Results of Library Construction

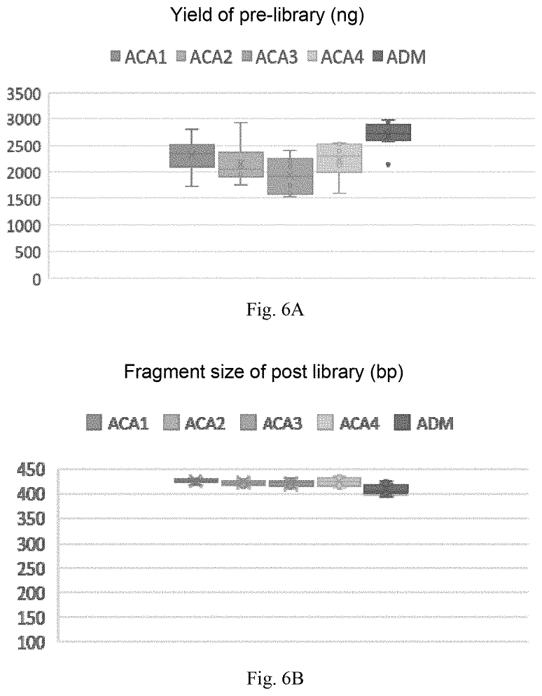

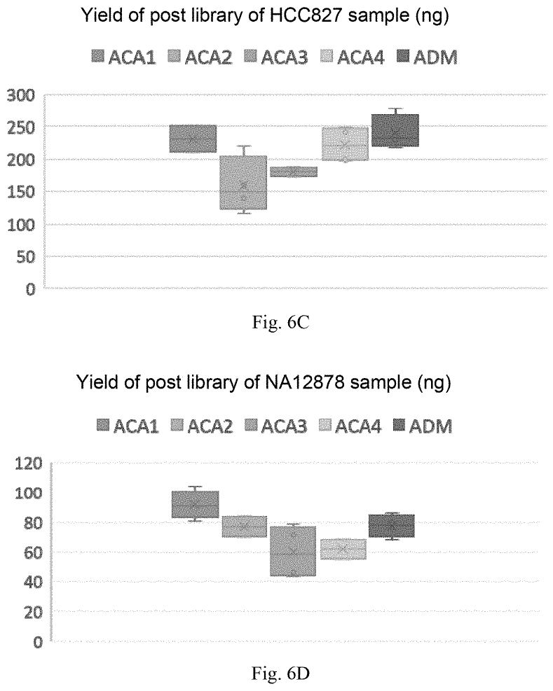

The QC index of library construction in the present disclosure focuses on pre-library yield, post library yield, and average fragment size of the post library. For detailed QC results and statistics, see table 18 below, and for statistical information, see FIGS. 6A-6D.

TABLE 18
QC results of library construction

| | | | | Fragment |
|---|---|---|---|---|
| | Well | Yield of pre- | Yield of final- | size of final- |
| Sample name | position | library(ng) | library(ng) | library(bp) |
| NA12878-ACA1 | A1 | 2412 | 91.2 | 425 |
| HCC827-ACA2 | B1 | 2001.6 | 220.2 | 430 |
| NA12878-ACA3 | C1 | 1742.4 | 46.02 | 416 |
| HCC827-ACA4 | D1 | 2390.4 | 198 | 420 |
| NA12878-ACA1 | E1 | 2800.8 | 81 | 429 |
| HCC827-ACA2 | F1 | 1756.8 | 138.6 | 418 |
| NA12878-ACA3 | G1 | 2203.2 | 71.4 | 423 |
| HCC827-ACA4 | H1 | 2545.2 | 248.4 | 431 |
| HCC827-ACA3 | A2 | 1530 | 172.8 | 431 |
| NA12878-ACA4 | B2 | 2124 | 68.4 | 415 |
| HCC827-ACA1 | C2 | 2260.8 | 211.2 | 426 |
| NA12878-ACA2 | D2 | 2174.4 | 70.2 | 416 |
| HCC827-ACA3 | E2 | 2095.2 | 187.2 | 424 |
| NA12878-ACA4 | F2 | 1598.4 | 55.8 | 427 |
| HCC827-ACA1 | G2 | 2422.8 | 250.8 | 426 |
| NA12878-ACA2 | H2 | 2088 | 84 | 425 |
| NA12878-ACA1 | A3 | 2221.2 | 104.4 | 430 |
| HCC827-ACA2 | B3 | 1947.6 | 159 | 421 |
| NA12878-ACA3 | C3 | 1598.4 | 43.32 | 410 |
| HCC827-ACA4 | D3 | 2199.6 | 199.2 | 416 |
| NA12878-ACA1 | E3 | 1728 | 90 | 418 |
| HCC827-ACA2 | F3 | 2926.8 | 117 | 423 |
| NA12878-ACA3 | G3 | 2397.6 | 79.2 | 419 |
| HCC827-ACA4 | H3 | 2520 | 241.2 | 432 |
| HCC827-ADM | A4 | 2754 | 278.4 | 426 |
| NA12878-ADM | B4 | 2930.4 | 79.8 | 394 |
| HCC827-ADM | C4 | 2685.6 | 229.8 | 400 |
| NA12878-ADM | D4 | 2671.2 | 86.4 | 402 |
| HCC827-ADM | E4 | 2980.8 | 235.2 | 420 |
| NA12878-ADM | F4 | 2563.2 | 76.2 | 406 |
| HCC827-ADM | G4 | 2750.4 | 217.8 | 403 |
| NA12878-ADM | H4 | 2145.6 | 68.4 | 412 |

The results show that the criteria for library construction of the contamination-resistant ACA adapter are lower than that of original ADM adapter in terms of pre-library yield. With the corresponding pre-library put to hybridization capture, the post library yields obtained from different types of samples in both experimental and control groups are similar, but the average fragment size of the post library is slightly larger than that of the control group.

This demonstrates that contamination-resistant ACA adapter can meet the QC index for normal library construction, but the effect in library construction is slightly lower than that of the original ADM adapter.

### Bioinformative QC Results of Sequencing

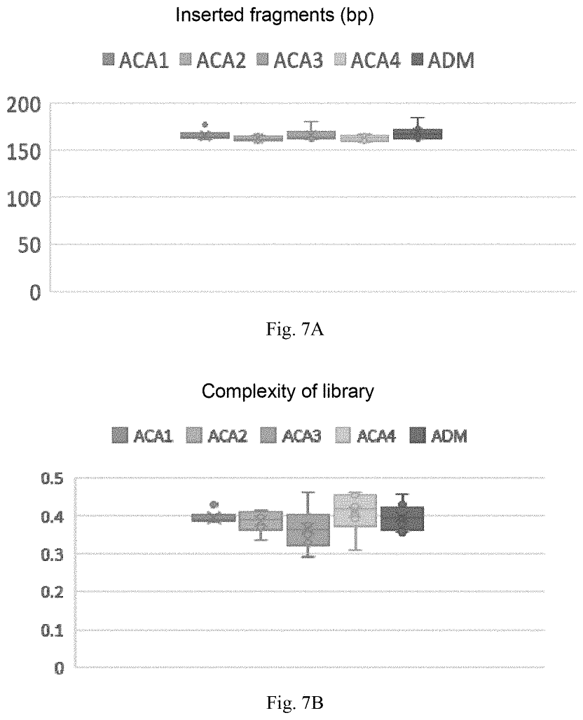

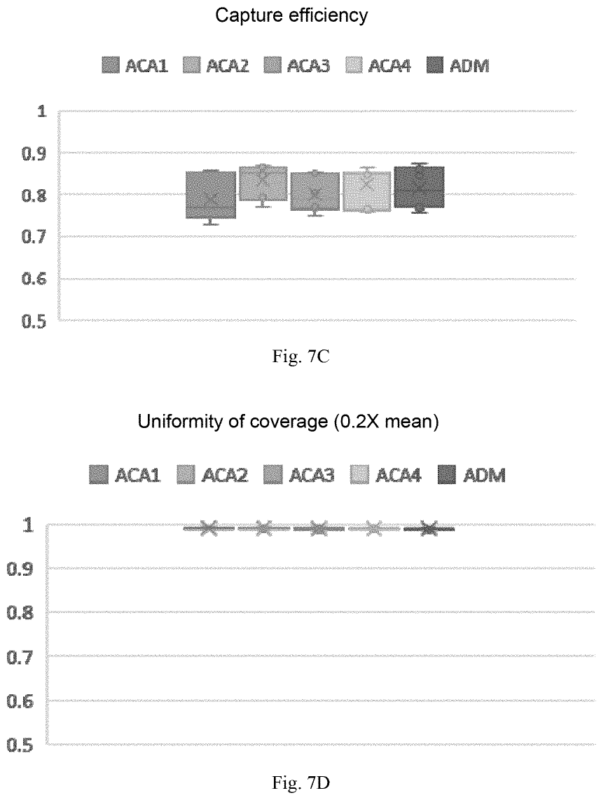

The QC index of targeted capture experiment in this example mainly focuses on insert size, capture efficiency, complexity of library construction and uniformity of coverage (0.2× mean). For detailed QC results, see table 19 below, and the statistical information is shown in FIGS. 7A-7D.

TABLE 19
Bioinformative QC of Sequencing

| | | Fragment | | | Uniformity |
|---|---|---|---|---|---|
| | | size of | Complexity | | of coverage |
| | Well | final-library | of library | Capture | (0.2 X |
| Sample name | position | (bp) | construction | efficiency | mean) |
| NA12878-ACA1 | A1 | 165 | 0.394 | 0.731 | 0.992 |
| HCC827-ACA2 | B1 | 160 | 0.396 | 0.844 | 0.992 |
| NA12878-ACA3 | C1 | 162 | 0.33 | 0.749 | 0.992 |
| HCC827-ACA4 | D1 | 159 | 0.407 | 0.851 | 0.991 |
| NA12878-ACA1 | E1 | 177 | 0.384 | 0.749 | 0.991 |
| HCC827-ACA2 | F1 | 161 | 0.382 | 0.856 | 0.992 |
| NA12878-ACA3 | G1 | 180 | 0.374 | 0.77 | 0.99 |
| HCC827-ACA4 | H1 | 167 | 0.424 | 0.845 | 0.99 |
| HCC827-ACA3 | A2 | 161 | 0.385 | 0.85 | 0.992 |
| NA12878-ACA4 | B2 | 163 | 0.391 | 0.759 | 0.991 |
| HCC827-ACA1 | C2 | 161 | 0.388 | 0.856 | 0.991 |
| NA12878-ACA2 | D2 | 165 | 0.369 | 0.77 | 0.991 |
| HCC827-ACA3 | E2 | 166 | 0.462 | 0.854 | 0.989 |
| NA12878-ACA4 | F2 | 165 | 0.369 | 0.77 | 0.991 |
| HCC827-ACA1 | G2 | 163 | 0.428 | 0.858 | 0.991 |
| NA12878-ACA2 | H2 | 166 | 0.41 | 0.793 | 0.991 |
| NA12878-ACA1 | A3 | 163 | 0.388 | 0.749 | 0.992 |
| HCC827-ACA2 | B3 | 160 | 0.413 | 0.87 | 0.99 |
| NA12878-ACA3 | C3 | 162 | 0.293 | 0.774 | 0.99 |
| HCC827-ACA4 | D3 | 160 | 0.464 | 0.866 | 0.99 |
| NA12878-ACA1 | E3 | 164 | 0.389 | 0.783 | 0.992 |
| HCC827-ACA2 | F3 | 161 | 0.338 | 0.865 | 0.991 |
| NA12878-ACA3 | G3 | 165 | 0.35 | 0.804 | 0.989 |
| HCC827-ACA4 | H3 | 163 | 0.453 | 0.853 | 0.99 |
| HCC827-ADM | A4 | 163 | 0.429 | 0.843 | 0.992 |
| NA12878-ADM | B4 | 167 | 0.393 | 0.758 | 0.989 |
| HCC827-ADM | C4 | 166 | 0.397 | 0.874 | 0.987 |
| NA12878-ADM | D4 | 185 | 0.357 | 0.768 | 0.991 |
| HCC827-ADM | E4 | 173 | 0.402 | 0.861 | 0.988 |
| NA12878-ADM | F4 | 162 | 0.358 | 0.774 | 0.991 |
| HCC827-ADM | G4 | 162 | 0.459 | 0.866 | 0.99 |
| NA12878-ADM | H4 | 168 | 0.376 | 0.774 | 0.99 |

The results show that contamination-resistant ACA adapters have no significant difference compared with the original ADM adapter and PPO primer in terms of insert size, capture efficiency, Complexity of library construction and uniformity of coverage (0.2× mean). This demonstrates that contamination-resistant ACA adapters can meet the requirement for bioinformative QC analysis of normal sequencing and has the same effect of analysis as that of the original ADM adapter.

### Mutation Detection Results

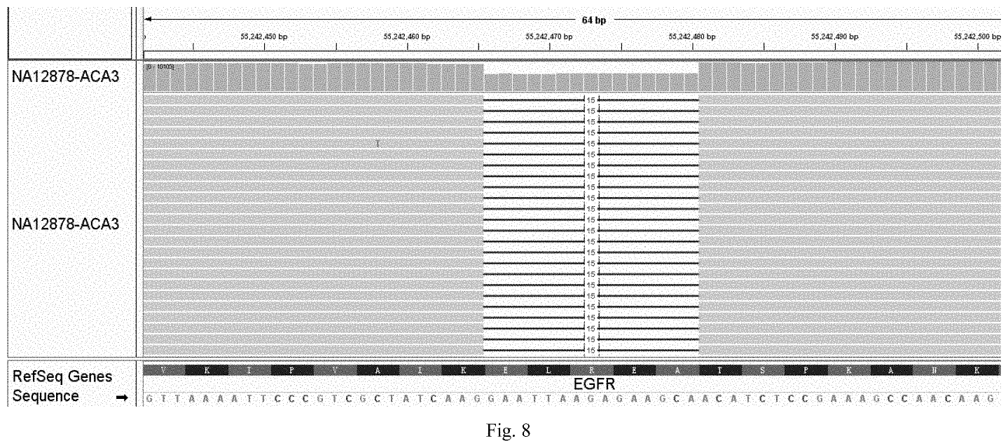

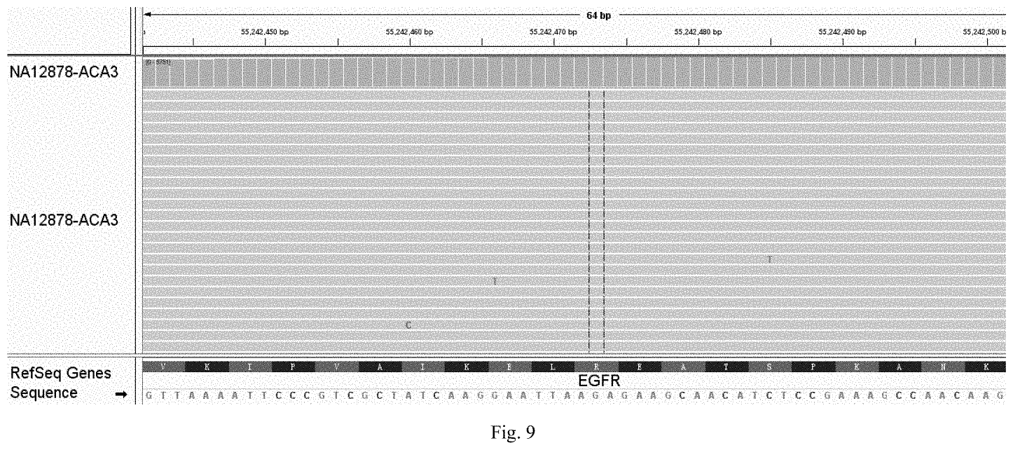

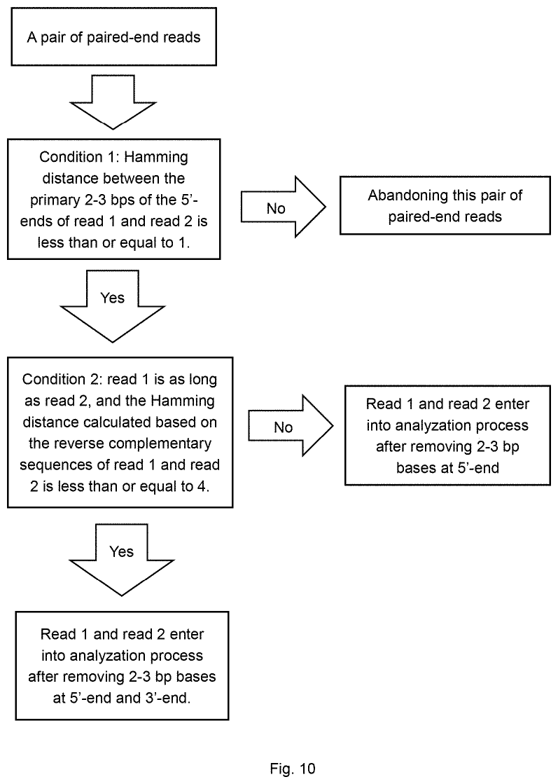

Processing the data using conventional data analysis procedure, mutation sites of positive samples that are manually introduced into the negative sample in adjacent wells in capture process are detected. The results are shown in detail in table 20, in which the EGFR A750del mutation site in the NA12878-ACA3 sample are as showed in FIG. 8.

In FIG. 8, NA12878 in wells G1, H2 and G3 are cross-contamination introduced manually, and NA12878 in well C3 is accidental real cross-contamination occurring in the experiment.

TABLE 20
Mutation Detection Results of processing
of conventional analysis procedure

| | Well | | |
|---|---|---|---|
| Sample name | position | EGFR: cn_amp | EGFR.p.E746_A750del |
| HCC827-ACA2 | B1 | 30.58 | 80.10% |
| HCC827-ACA4 | D1 | 29.97 | 80.42% |
| HCC827-ACA2 | F1 | 32.70 | 80.47% |
| NA12878-ACA3 | G1 | 5.82 | 31.60% |
| HCC827-ACA4 | H1 | 30.59 | 80.77% |
| HCC827-ACA3 | A2 | 34.30 | 81.31% |
| HCC827-ACA1 | C2 | 30.70 | 79.83% |
| HCC827-ACA3 | E2 | 32.14 | 81.01% |
| HCC827-ACA1 | G2 | 29.02 | 80.50% |
| NA12878-ACA2 | H2 | 6.98 | 41.54% |
| HCC827-ACA | B3 | 31.15 | 80.06% |
| NA12878-ACA3 | C3 | | 0.34% |
| HCC827-ACA4 | D3 | 31.92 | 81.00% |
| HCC827-ACA2 | F3 | 32.83 | 80.44% |
| NA12878-ACA3 | G3 | 7.54 | 39.52% |
| HCC827-ACA | H3 | 30.26 | 80.70% |
| HCC827-ACA | A4 | 37.13 | 82.65% |
| HCC827-ACA | C4 | 37.12 | 82.30% |
| HCC827-ACA | E4 | 36.28 | 83.44% |
| HCC827-ACA | G4 | 37.50 | 82.35% |

When mutation detection is re-performed on the data using the newly designed analysis procedure, mutations in positive sample manually introduced and really occurring in negative samples are successfully removed. The results are as in table 21, in which the detection results of the EGFR A750del site in NA12878-ACA3 sample are shown in FIG. 8.

TABLE 21
Mutation Detection Results of newly designed analysis procedure

| | Well | | |
|---|---|---|---|
| Sample name | position | EGFR: can_amp | EGFR.p.E746_A750del |
| HCC827-ACA2 | B1 | 37.17 | 81.87% |
| HCC827-ACA4 | D1 | 36.16 | 82.32% |
| HCC827-ACA2 | F1 | 38.83 | 82.18% |
| HCC827-ACA4 | H1 | 36.92 | 82.48% |
| HCC827-ACA3 | A2 | 39.52 | 82.56% |
| HCC827-ACA1 | C2 | 37.79 | 81.42% |
| HCC827-ACA3 | E2 | 38.48 | 82.26% |
| HCC827-ACA1 | G2 | 36.41 | 82.07% |
| HCC827-ACA2 | B3 | 37.71 | 81.85% |
| HCC827-ACA4 | D3 | 37.71 | 81.85% |
| HCC827-ACA2 | F3 | 38.74 | 82.29% |
| HCC827-ACA4 | H3 | 36.54 | 82.51% |
| HCC827-ADM | A4 | 37.13 | 82.65% |
| HCC827-ADM | C4 | 37.12 | 82.30% |
| HCC827-ADM | E4 | 36.28 | 83.44% |
| HCC827-ADM | G4 | 37.5 | 82.35% |

Statistical analysis performed on the processed data shows that the use contamination-resistant ACA adapter combined with the newly designed bioinformative analysis procedure, effectively avoids interference of the manually introduced and really occurring mutation in positive samples on the detection. The detailed statistical results are as in table 22 below.

TABLE 22
Statistics of mutation sites after processing of newly designed analysis procedure

| | | EGFR. A750del | | | EGFR: cn_amp | |
|---|---|---|---|---|---|---|
| | | Sequencing | Total depth | | Copy | Sequencing depth |
| | Well | Depth of site | of site | mutation | number of | of deduplication |
| Sample name | position | mutation | sequencing | abundance | mutation | of sample |
| HCC827-1 | B1 | 21,732 | 24,796 | 81.87% | 37.17 | 1,798 |
| HCC827-2 | D1 | 24,568 | 28,072 | 82.32% | 36.16 | 2,099 |
| HCC827-3 | F1 | 17,168 | 19,584 | 82.18% | 38.83 | 1,399 |
| HCC827-4 | H1 | 24,259 | 27,666 | 82.48% | 36.92 | 2,032 |
| HCC827-5 | A2 | 16,990 | 19,413 | 82.56% | 39.52 | 1,333 |
| HCC827-6 | C2 | 21,013 | 24,024 | 81.42% | 37.79 | 1,723 |
| HCC827-7 | E2 | 20,363 | 23,242 | 82.26% | 38.48 | 1,646 |
| HCC827-8 | G2 | 24,002 | 27,323 | 82.07% | 36.41 | 2,035 |
| HCC827-9 | B3 | 20,205 | 23,071 | 81.85% | 37.71 | 1,654 |
| HCC827-10 | D3 | 21,362 | 24,270 | 82.77% | 38.09 | 1,772 |
| HCC827-11 | F3 | 16,394 | 18,704 | 82.29% | 38.74 | 1,347 |
| HCC827-12 | H3 | 24,249 | 27,684 | 82.51% | 36.54 | 2,112 |
| HCC827-13 | A4 | 23,286 | 26,511 | 82.65% | 37.13 | 1,896 |
| HCC827-14 | C4 | 24,663 | 28,202 | 82.30% | 37.12 | 2,008 |
| HCC827-15 | E4 | 25,290 | 28,794 | 83.44% | 36.28 | 2,083 |
| HCC827-16 | G4 | 22,454 | 25,591 | 82.35% | 37.50 | 1,888 |
| NA12878-1 | A1 | 0 | 2,361 | NA | NA | 2,540 |
| NA12878-2 | C1 | 0 | 1,655 | NA | NA | 1,728 |
| NA12878-3 | E1 | 0 | 2,579 | NA | NA | 2,669 |
| NA12878-4 | G1 | 0 | 2,024 | NA | NA | 2,225 |
| NA12878-5 | B2 | 0 | 2,420 | NA | NA | 2,587 |
| NA12878-6 | D2 | 0 | 2,468 | NA | NA | 2,628 |
| NA12878-7 | F2 | 0 | 1,716 | NA | NA | 1,810 |
| NA12878-8 | H2 | 0 | 2,771 | NA | NA | 2,884 |
| NA12878-9 | A3 | 0 | 2,475 | NA | NA | 2,618 |
| NA12878-10 | C3 | 0 | 1,447 | NA | NA | 1,563 |
| NA12878-11 | E3 | 0 | 2,923 | NA | NA | 3,048 |
| NA12878-12 | G3 | 0 | 2,365 | NA | NA | 2,476 |
| NA12878-13 | B4 | 0 | 2,611 | NA | NA | 2,755 |
| NA12878-14 | D4 | 0 | 2,845 | NA | NA | 2,903 |
| NA12878-15 | F4 | 0 | 2,300 | NA | NA | 2,467 |
| NA12878-16 | H4 | 0 | 2,255 | NA | NA | 2,313 |

It demonstrates that the newly designed contamination-resistant ACA adapters combined with corresponding bioinformative analysis procedure can effectively avoid the generation of erroneous experimental data resulted from cross-contamination of samples caused by external factors in the experiment, thereby further improving the accuracy of the experiment.

### Illustrations of Bioinformative Analysis

After the sequencing data is off-lined, if the first but not the second of the following two conditions is met, it is considered that a pair of reads possesses a contamination-resistant adapter at the 5′-end, and no contamination-resistant adapter at the 3′-end; if the following two conditions are both met, it is considered that a pair of reads possesses contamination-resistant adapters at both the 5′- and the 3′-ends.
Condition 1: Calculating Hamming distance between the primary 2-3 bps of the 5′-end of a pair of reads with same sequence ID, i.e., the read 1 sequence and the read 2 sequence respectively and the primary 2-3 bps of the 5′-end of the contamination-resistant adapter (A7), and the sum of numerical values is less than or equal to 1;
Condition 2: In the case that condition 1 is met and a pair of reads are of equal length, the reverse complementary sequence of one read is approximately the same as the forward sequence of the other read, that is, Hamming distance calculated with the characters of sequence of the two reads is less than or equal to the default value 4 set by the software.

In the process of subsequent analysis, for a pair of reads with contamination-resistant adapter-specific sequences merely at the 5′-end, only the 2-3 bps at the 5′-end of the read are subtracted; and for the 5′ a pair of reads with contamination-resistant adapter-specific sequences at both the 5′- and the 3′-end, the 2-3 bps at both the 5′-end and 3′-end of the read are subtracted. Put a pair of reads after the above two types of subtraction of contamination-resistant adapter specific sequences in the fastq files of retained read 1 and read 2; and for a pair of reads that do not meet condition 1, it is put in the fastq files of abandoned read 1 and read 2, for subsequent inspection and analysis.

The software will judge the type of contamination-resistant adapter of off-line raw fastq file, and giving the proportion of the judged adapter sequences and the type of dominant adapter during the analysis; if the proportion of type of the dominant adapter is less than 90%, it is considered that the sample has been contaminated with other samples, and the subsequent analysis procedures are stopped. If the dominant adapter type accounts for more than 90% but less than 98%, it is considered that the sample has been slightly contaminated with other samples, and the subsequent analysis procedures can be performed after removing the reads containing the contaminated adapter; If the dominant adapter type accounts for more than 98%, it is considered that the sample are not contaminated, and the subsequent analysis procedures are carried out directly. The total number of read pairs of the original data file, the number of read pairs whose adapter are cleaved, the number of read pairs eventually retained and the number of abandoned read pairs are counted in the final results of analysis.

TABLE 23
A pair of contamination-resistant adapters that are removed of 5′-
and the 3′-ends respectively

| Type of contamination- | |
|---|---|
| resistant adapters | Sequence |
| 5′-end contamination- | AT |
| resistant adapter | |
| 3′-end contamination- | AT |
| resistant adapter (reversely | |
| complemented sequence of | |
| 5′-end contamination- | |
| resistant adapter) | |
| Read 1 sequence | ATGTAAATGCACAACAGTGAGACGCAG |
| | AATGCCTCTGGAGCACACAGAAGGGAC |
| | GCCTCATCCAGAGCTGGGGGATTAGAGA |
| | AGGCTCCCAGAAGTGAAATTAGCTGAT |
| Read 2 sequence | ATCAGCTAATTTCACTTCTGGGAGCCTT |
| | CTCTAATCCCCCAGCTCTGGATGAGGCG |
| | TCCCTTCTGTGTGCTCCAGAGGCATTCT |
| | GCGTCTCACTGTTGTGCATTTACAT |
| Reversely complemented | ATGTAAATGCACAACAGTGAGACGCAG |
| sequence of read 2 | AATGCCTCTGGAGCACACAGAAGGGAC |
| | GCCTCATCCAGAGCTGGGGGATTAGAGA |
| | AGGCTCCCAGAAGTGAAATTAGCTGAT |
| Read 1 sequence removed of | ATGTAAATGCACAACAGTGAGACGCAG |
| “AT” at the 3′-end | AATGCCTCTGGAGCACACAGAAGGGAC |
| | GCCTCATCCAGAGCTGGGGGATTAGAGA |
| | AGGCTCCCAGAAGTGAAATTAGCTG |
| Read 2 sequence removed of | ATCAGCTAATTTCACTTCTGGGAGCCTT |
| “AT” at the 3′-end | CTCTAATCCCCCAGCTCTGGATGAGGCG |
| | TCCCTTCTGTGTGCTCCAGAGGCATTCT |
| | GCGTCTCACTGTTGTGCATTTAC |
| Read 1 sequence removed of | GTAAATGCACAACAGTGAGACGCAGAA |
| “ATs” at both the 3′-end and | TGCCTCTGGAGCACACAGAAGGGACGC |
| the 5′-end | CTCATCCAGAGCTGGGGGATTAGAGAA |
| | GGCTCCCAGAAGTGAAATTAGCTG |
| Read 2 sequence removed of | CAGCTAATTTCACTTCTGGGAGCCTTCT |
| “ATs” at both the 3′-end and | CTAATCCCCCAGCTCTGGATGAGGCGTC |
| 5′-end | CCTTCTGTGTGCTCCAGAGGCATTCTGC |
| | GTCTCACTGTTGTGCATTTAC |

Notes:
In table 23, both the 5′-end and the 3′-end of contamination-resistant adapters are “AT”, the lengths of read 1 and 2 are the same, read 1 and 2 are reversely complementary to each other, so the contamination-resistant adapters “AT” at the 5′-end and 3′-end of this pair of reads are subtracted during the analysis.

TABLE 24
A pair of contamination-resistant adapters that are removed of 5′-end
respectively

| Type of contamination- | |
|---|---|
| resistant adapters | Sequence |
| 5′-end contamination- | TCT |
| resistant adapter | |
| 3′-end contamination- | AGA |
| resistant adapter | |
| (reversely complemented | |
| sequence of 5′-end | |
| contamination-resistant | |
| adapter) | |
| Read 1 sequence | TCTAATGGACAAATAAAAGTTGTATATATTTA |
| | CTGTATACAACACGATGTTTTGGAATATGTAT |
| | ACGTTGTGGAATGGCTAAATCAAGCTAATTA |
| | AAATATGCATTACTTCACTTTTTTTTTTTTTA |
| | AGAGACAGCGTTTTGCTCTCGTT |
| Read 2 sequence | TCTTGATGCAGTGAGCCGAGATCATGCCACT |
| | TCTGTCTCTTAAAAAAAAAAAAAGTGTAGT |
| | AATGCATATTTTAATTATCTTGATTTATCATTT |
| | CTACAATGTAGTCCTATTCCAAAGTAT |
| Reversely complemented | ATACTTTGGAATAGGACTACATTGTAGAAAT |
| sequence of read | GATAAATCAAGATAATTAAAATATGCATTACT |
| | ACACTTTTTTTTTTTTTAAGAGACAGAGTTT |
| | TGCTCTCGTTACCCAGGCTGGAGTACAGTG |
| | GCATGATCTCGGCTCACTGCATCAAGA |
| Read 1 sequence removed | AATGGACAAATAAAAGTTGTATATATTTACTG |
| of “TCT” at the 5′-end | TATACAACACGATGTTTTGGAATATGTATACG |
| | TTGTGGAATGGCTAAATCAAGCTAATTAAAA |
| | TATGCATTACTTCACTTTTTTTTTTTTTAAGA |
| | GACAGCGTTTTGCTCTCGTT |
| Read 2 sequence removed | TGATGCAGTGAGCCGAGATCATGCCACTGTA |
| of “TCT” at the 5′-end | CTCCAGCCTGGGTAACGAGAGCAAAACTCT |
| | GTCTCTTAAAAAAAAAAAAAGTGTAGTAAT |
| | GCATATTTTAATTATCTTGATTTATCATTTCTA |
| | CAATGTAGTCCTATTCCAAAGTAT |

Notes:
In table 24, the 5′-end of contamination-resistant adapters is “TCT”, and the 3′-end of contamination-resistant adapters is “AGA”, the lengths of read 1 and 2 are not the same, reversely complementary sequence of read 2 is different from that of read 1, so the contamination-resistant adapters “TCT” at the 5′-end of this pair of reads are subtracted during the analysis.

TABLE 25
A pair of reads that are not removed of 5′-end and abandoned

| Type of contamination- | |
|---|---|
| resistant adapters | Sequence |
| 5′-end contamination- | GT |
| resistant adapter | |
| Read 1 sequence | GGATAGAGGGGCACCACGTTCTTGCACTTC |
| | ATGCTGTACAGATGCTCCATTCCTTTGTTACT |
| | GTAGGTGGGAAGACACAGAAAGGACTACTT |
| | TAGAGCCAACCCGAGCCCCAGGAGTGCTGA |
| | AATCCCTAGAAGGGGAAGGAACAGGAACG |
| Read 2 sequence | GAGTGTCTTTGGAGTTCCTCTTCCTACCCCT |
| | TCTAGGGATTTCAGCACTCCTGGGGCTCGGG |
| | TTGGCACTAAAGTATTCCTTACTGTGACTTC |
| | CCACCTACACTAACAAAGGCAACGAGCATC |
| | TTTACCGCATGAAGTGCAAGAACGAGGG |

Notes:
In table 25, the 5′-end of the contamination-resistant adapters is “GT”, and the first two bases at 5′-end of read 1 and 2 are “GG” and “GA”; the sequences are different from the contamination-resistant adapters by one base, the sum of numerical values of Hamming distance calculated between “GG” and “GA” respectively and “GT” is 2, which is bigger than 1, so this pair of reads is abandoned.

1. A method for preparing a DNA library, comprising a pre-library preparation process, wherein the pre-library preparation process comprising DNA preparation, end repairing and 3′A tailing, adapter ligation using contamination-resistant adapters, purification of adapter ligation products, amplification of pre-library, and purification of the amplified pre-library, wherein the contamination-resistant adapters are additionally added with 2-3 bps at the 3′-end or 5′-end compared with original adapters used to prepare the DNA library, thus forming multiple pairs of contamination-resistant adapters, the multiple pairs of contamination-resistant adapters are preferably 4, 5, 6, 7 or 8 pairs.

2. Method for preparing a DNA library according to claim 1, wherein the contamination-resistant adapters are designed to meet following criteria:
(1) adding bases from the 3′-end of the original adapter, and ensuring that the last base added is a T;
(2) adding A, T, G and C to the first position from the 3′-end of the original adapter to ensure signal equilibrium during sequencing and no effect on the judgment about detected bases;
(3) on each position added at the 3′-end of the original adapter, the proportion of the same bases does not exceed 50%;
following (1)-(3) above, multiple first contamination-resistant adapters are obtained;
and
(4) at the 5′-end of the original adapter adding the bases that are reversely complementary to the extra bases except terminal T in the first contamination-resistant adapters, and the first base at the 5′-end is phosphorylated, thus multiple second contamination-resistant adapters are obtained.

3. Method of preparation according to claim 1 or 2, wherein on the position of the first proximal base added at the 3′-end of the original adapter, there are 4 types of bases, each accounting for 25%; on the position of the second proximal base added, there are 3 types of bases, with T bases accounting for 50%, and the remaining 2 types of bases each accounting for 25%; at the position of the third proximal base added at the 3′-end or 5′-end of the original adapter, there is no base for two adapters, and a fixed base T for the other two adapters, accounting for 50%.

4. Method of preparation according to any one of the precedent claims, wherein the original adapters are:
ADM-A5:
ACACTCTTTCCCTACACGACGCTCTTCCGATC*T
ADM-A7:
/5Phos/GATCGGAAGAGCACACGTCTGAACTCCAGTCAC;
*represents phosphorothioate-modification; /5Phos/ represents phosphorylation modification.

5. Method of preparation according to any one of the precedent claims, wherein for multiple first contamination-resistant adapters, the extra base sequences are A*T, G*T, TC*T and CA*T; and for multiple second contamination-resistant adapters, the additional bases are TA, CA, GAA and TGA, * represents phosphorothioate-modification; /5Phos/ represents phosphorylation modification.

6. Method of preparation according to any one of the precedent claims, wherein the contamination-resistant adapters are:
ACA1-A5:
ACACTCTTTCCCTACACGACGCTCTTCCGATCTA*T
ACA1-A7:
/5Phos/TAGATCGGAAGAGCACACGTCTGAACTCCAGTCAC
ACA2-A5:
ACACTCTTTCCCTACACGACGCTCTTCCGATCTG*T
ACA2-A7:
/5Phos/CAGATCGGAAGAGCACACGTCTGAACTCCAGTCAC
ACA3-A5:
ACACTCTTTCCCTACACGACGCTCTTCCGATCTTC*T
ACA3-A7:
/5Phos/GAAGATCGGAAGAGCACACGTCTGAACTCCAGTCAC
ACA4-A5:
ACACTCTTTCCCTACACGACGCTCTTCCGATCTCA*T
ACA4-A7:
/5Phos/TGAGATCGGAAGAGCACACGTCTGAACTCCAGTCAC
*represents phosphorothioate-modification; /5Phos/ represents phosphorylation modification, wherein the bases that are underlined and bolded are extra bases.

7. Method of preparation according to any one of the precedent claims, wherein test samples are arranged such that each of the contamination-resistant adapter is different from those at adjacent or surrounding locations.

8. Method of preparation according to any one of the precedent claims, wherein the following primers are used for pre-library amplification:
Oligo PPS 1.1:
ACACTCTTTCCCTACACGACGCTC;
Oligo PPS 2.1:
GTGACTGGAGTTCAGACGTGTGC

9. Method of preparation according to claim 6, wherein the test samples are arranged such that basic arrangement units of the contamination-resistant adapter are:

| | |
|---|---|
| ACA1 | ACA3 |
| ACA2 | ACA4 |
| ACA3 | ACA1 |
| ACA4 | ACA2; |

wherein ACA1 means by using ACA1-A5 and ACA1-A7, ACA2 means by using ACA2-A5 and ACA2-A7, ACA3 means by using ACA3-A5 and ACA3-A7, and ACA4 means by using ACA4-A5 and ACA4-A7.

10. Use of contamination-resistant adapter according to any one of the precedent claims in preparation of a DNA library capture kit.

11. Use according to claim 10, wherein the DNA library is a cfDNA library, a leukocyte gDNA library or a tissue-derived DNA library.

12. Method for performing bioinformative analysis of DNA library prepared by preparation methods according to any one of the claims 1-9, comprising sequencing and analyzing sequencing data; if Condition 1 but not Condition 2 of the following two conditions is met, it is deemed that a pair of reads possesses a contamination-resistant adapter at the 5′-end, and no contamination-resistant adapter at the 3′-end; if the following two conditions are both met, it is deemed that a pair of reads possesses contamination-resistant adapters at both the 5′-end and the 3′-end;
Condition 1: calculating Hamming distance between the primary 2-3 bps of the 5′-end of a pair of reads with same sequence ID, i.e., the read 1 sequence and the read 2 sequence respectively and the 2-3 bps of the 5′-end of the contamination-resistant adapter, and the sum of numerical values is less than or equal to 1;
Condition 2: in the case that condition 1 is met, and a pair of reads are of equal length, the reverse complementary sequence of one read is approximately the same as the forward sequence of the other read, that is, Hamming distance calculated with the sequence characters of the two reads is less than or equal to the default value 4 set by the software.

13. Method according to claim 12, wherein in the subsequent analysis process, for a pair of reads with contamination-resistant adapter-specific sequences merely at the 5′-end, only the 2-3 bps at the 5′-end of the read are subtracted; and for a pair of reads with contamination-resistant adapter-specific sequences at both the 5′-end and the 3′-end, the 2-3 bps at both the 5′-end and 3′-end of the read are subtracted.

14. Method according to claim 13, wherein a pair of reads after the two deduction of contamination-resistant adapters are put respectively in the fastq files of the retained read 1 and read 2; and for a pair of reads that do not meet condition 1, the pair of reads are put in the fastq files of abandoned read 1 and read 2 for subsequent inspection and analysis.

15. Method according to any one of claims 12-14, comprising judging the type of the contamination-resistant adapters, and giving the proportion of the judged adapters and the type of dominant adapters during the analysis; if the proportion of the dominant adapter type is less than 90%, it is deemed that the sample has been contaminated with other samples, and the subsequent analysis procedures are stopped; if the dominant adapter type accounts for more than 90% but less than 98%, it is deemed that the sample has been slightly contaminated with other samples, and the subsequent analysis procedures can be performed after removing the reads containing the contaminated adapter; if the dominant adapter type accounts for more than 98%, it is deemed that the sample are not contaminated, and the subsequent analysis procedures are directly carried out.

16. Method according to any one of claims 12-15, wherein the total number of read pairs of the original data file, the number of read pairs whose adapter are cleaved, the number of read pairs eventually retained and the number of abandoned read pairs are counted in the final analysis results.
